(19) United States
(12) Patent Application Publication
(10) Pub. No.: US 2025/0166224 A1
(43) Pub. Date: May 22, 2025

(54) REGISTRATION OF DEFORMABLE STRUCTURES

(71) Applicant: The Johns Hopkins University, Baltimore, MD (US)

(72) Inventors: Jeffrey H. SIEWERDSEN, Baltimore, MD (US); Rohan VIJAYAN, Baltimore, MD (US)

(73) Assignee: The Johns Hopkins University, Baltimore, MD (US)

(21) Appl. No.: 18/839,298

(22) PCT Filed: Feb. 21, 2023
(86) PCT No.: PCT/US2023/013495

§ 371 Date: Aug. 16, 2024

Related U.S. Application Data
(60) Provisional application No. 63/312,690, filed on Feb. 22, 2022.

(51) Int. Cl. G06T 7/73; G06T 7/00

(52) U.S. Cl. CPC G06T 7/74; G06T 7/0014; G06T 2207/10081; G06T 2207/10124; G06T 2207/20016; G06T 2207/20021; G06T 2207/30008

(57) ABSTRACT

A 2D x-ray system includes an x-ray illumination system constructed to illuminate an object with an x-ray beam, a detection system arranged to receive at least a portion of said x-ray beam after passing through at least a portion of said object, and a system for registering a 2D x-ray image to a 3D x-ray image. The system includes a computer processor and computer memory that includes non-transient code, which when executed by said computer processor causes the computer processor to perform a method that includes generating multiple 2D projection images from a 3D x-ray image of the object, and registering said 2D x-ray image to one of said 2D projection images. Said registering uses a similarity metric that is differentially weighted to affect an influence of at least one of low-contrast or high-contrast structures on the registering between said 2D x-ray image and each of said 2D projection images.

### CROSS-REFERENCE OF RELATED APPLICATION

This application claims priority to U.S. Provisional Application No. 63/312,690, filed Feb. 22, 2022, which is incorporated herein by reference in its entirety.

### BACKGROUND

### 1. Technical Field

The currently claimed embodiments of the present invention relate to 3D-2D registration, and more particularly to 3D-2D registration of low contrast and/or deformable structures.

### 2. Discussion of Related Art

Existing 3D-2D registration methods are primarily used to resolve the pose of rigid, strongly attenuating objects that present strong gradients in an x-ray projection. Examples of such objects include bones, wires, surgical instruments, and robotic effectors. The strong gradients present in the x-ray projection encourage the use of gradient-based optimization methods that compare digitally reconstructed radiographs (DRRs) to the projection and iteratively resolve the pose of the object using gradient-based similarity metrics. Example metrics demonstrated for 3D-2D registration include gradient orientation (GO), gradient correlation (GC), and gradient information (GI).

3D-2D registration can be challenged to register anatomical structures that do not present strong gradients in the x-ray projection-especially when such structures are superimposed with other, higher contrast structures- and especially when such structures do not obey a 6 degree-of-freedom (DoF) rigid transformation between the 3D and 2D images (e.g. deformable structures, such as soft tissues). An important example is thoracic imaging, where low-contrast, deformable soft tissues (lung parenchyma and airways) are superimposed by high-contrast structures (ribs), and it is the low-contrast tissues that are the structures of interest.

In pulmonary interventions, for example, intraoperative CBCT/fluoroscopy are increasingly used to localize lung nodules within low-contrast airways. 3D-2D registration would be useful to compensate for voluntary or involuntary motion and deformation; however, conventional 3D-2D registration methods fail in this context for two reasons: (1) conventional methods would be driven by strong image gradients presented by ribs and thereby misregister the soft-tissue structures of interest; and (2) conventional methods assume a rigid motion model (up to 6 degrees of freedom in the resulting transform) and would not account for soft-tissue deformation. Therefore, there remains a need for improved 3D-2D registration of low-contrast and/or deformable structures.

### SUMMARY

An embodiment of the present invention is a method of registering a two-dimensional (2D) x-ray image to a three-dimensional (3D) x-ray image. The method includes: constructing said 2D x-ray image of an object of interest: receiving said 3D x-ray image of said object of interest: generating a plurality of 2D projection images from said 3D x-ray image of said object of interest for each of a corresponding plurality of different poses of said object of interest; and registering said 2D x-ray image to one of said 2D projection images. The registering uses a similarity metric that is differentially weighted to affect an influence of at least one of low-contrast or high-contrast structures on the registering between said 2D x-ray image and each of said 2D projection images.

Another embodiment of the present invention is a computer-readable medium for registering a two-dimensional (2D) x-ray image to a three-dimensional (3D) x-ray image comprising non-transient code, which when executed by a computer causes the computer to perform a method that includes: constructing said 2D x-ray image of an object of interest; receiving said 3D x-ray image of said object of interest: generating a plurality of 2D projection images from said 3D x-ray image of said object of interest for each of a corresponding plurality of different poses of said object of interest; and registering said 2D x-ray image to one of said 2D projection images. The registering uses a similarity metric that is differentially weighted to affect an influence of at least one of low-contrast or high-contrast structures on the registering between said 2D x-ray image and each of said 2D projection images.

Another embodiment of the present invention is a two-dimensional (2D) x-ray system. The system includes: an x-ray illumination system constructed to illuminate an object of interest with an x-ray beam: a detection system arranged to receive at least a portion of said x-ray beam after said x-ray beam after passing through at least a portion of said object of interest; and a system for registering a two-dimensional (2D) x-ray image to a three-dimensional (3D) x-ray image, said system comprising: a computer processor and computer memory. The computer memory comprises non-transient code, which when executed by said computer processor causes the computer processor to perform a method that includes: constructing said 2D x-ray image of an object of interest: receiving said 3D x-ray image of said object of interest: generating a plurality of 2D projection images from said 3D x-ray image of said object of interest for each of a corresponding plurality of different poses of said object of interest; and registering said 2D x-ray image to one of said 2D projection images. The registering uses a similarity metric that is differentially weighted to affect an influence of at least one of low-contrast or high-contrast structures on the registering between said 2D x-ray image and each of said 2D projection images.

### BRIEF DESCRIPTION OF THE DRAWINGS

Further objectives and advantages will become apparent from a consideration of the description, drawings, and examples.

FIG. 1 shows a two-dimensional (2D) x-ray system of some embodiments.

FIG. 2 shows a process of some embodiments for registering a two-dimensional (2D) x-ray image to a three-dimensional (3D) x-ray image.

FIG. 3 illustrates a process of some embodiments for 3D-2D registration, suitable for rigid registration of high-contrast structures.

FIG. 4 illustrates a limitation in some embodiments of some 3D-2D registration methods.

FIG. 5 shows a process of some embodiments and associated weighting maps for down-weighting of bone gradients.

FIG. 6A shows an implementation in some embodiments of a method for low-contrast 3D-2D registration.

FIG. 6B shows an alternate method of some embodiments for gradient down-weighting for suppressing the influence of bone gradients in 3D-2D registration.

FIG. 7 shows an example of some embodiments of low-contrast, locally rigid 3D-2D registration for a designated ROI in a chest radiograph.

FIG. 8 shows an example of some embodiments of multi-scale, locally-rigid 3D-2D registration combined with methods for low-contrast 3D-2D registration.

FIG. 9 shows an example of some embodiments of multi-scale, locally-rigid, globally deformable 3D-2D registration combined with methods for low-contrast 3D-2D registration, multi-scale locally rigid 3D-2D registration, applied to many ROIs.

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed, and other methods developed, without departing from the broad concepts of the current invention.

All references cited anywhere in this specification, including the Background and Detailed Description sections, are incorporated by reference as if each had been individually incorporated.

The term “differentially weighted” as used herein means that it is in some spatial distribution which can be, or can be thought of, as a “map”, for example.

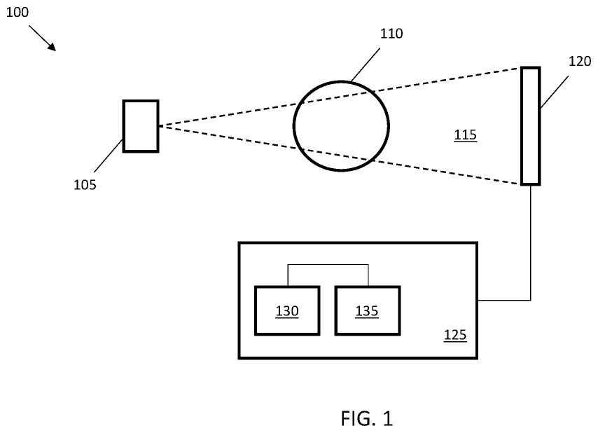

FIG. 1 shows a two-dimensional (2D) x-ray system 100 of some embodiments. The 2D x-ray system 100 may be any x-ray system, including but not limited to a fluoroscopy x-ray system, or a dual-energy x-ray system.

The system 100 includes an x-ray illumination system 105 constructed to illuminate an object of interest 110 with an x-ray beam 115. The system 100 also includes a detection system 120 arranged to receive at least a portion of the x-ray beam 115 after the x-ray beam 115 after passing through at least a portion of the object of interest 110.

The system 100 also includes a registration system 125 for registering a two-dimensional (2D) x-ray image to a three-dimensional (3D) x-ray image. The registration system 125 is communicatively coupled to the detection system 120, and includes a computer processor 130 communicatively coupled to a computer memory 135.

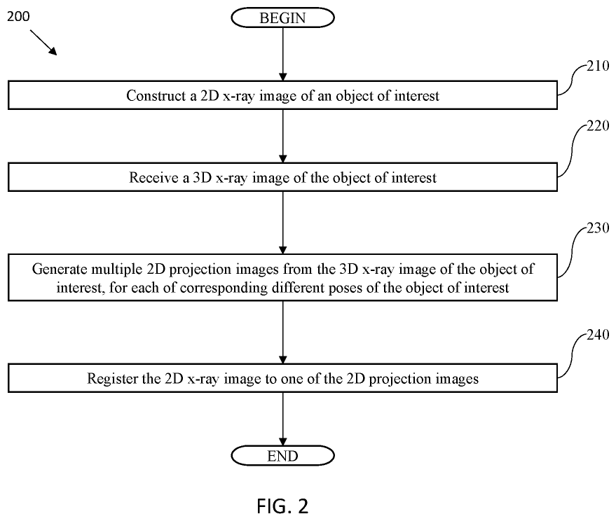

FIG. 2 shows a process 200 of some embodiments for registering a two-dimensional (2D) x-ray image to a three-dimensional (3D) x-ray image. The process 200 may be performed, for example, by the computer processor 130, which executes non-transient code stored in the computer memory 135.

The process 200 starts at 210, by constructing a 2D x-ray image of an object of interest. For example, the process 200 may receive imaging data from the detection system 120, and process the imaging data to construct the 2D x-ray image.

At 220, the process 200 receives a 3D x-ray image of the object of interest. In some embodiments, the 3D x-ray image may be received from a 3D imaging system (not shown) that is external to the 2D x-ray system 100. In some embodiments, the 3D x-ray image may be generated by a 3D x-ray system, such as but not limited to a cone-beam computed tomography (CBCT) system.

At 230, the process 200 generates multiple 2D projection images from the 3D x-ray image of the object of interest, for each of corresponding different poses of the object of interest.

At 240, the process 200 registers the 2D x-ray image to one of the 2D projection images. In some embodiments, the registering uses a similarity metric that is differentially weighted to affect an influence of at least one of low-contrast or high-contrast structures on the registering between the 2D x-ray image and each of the 2D projection images. The process 200 then ends.

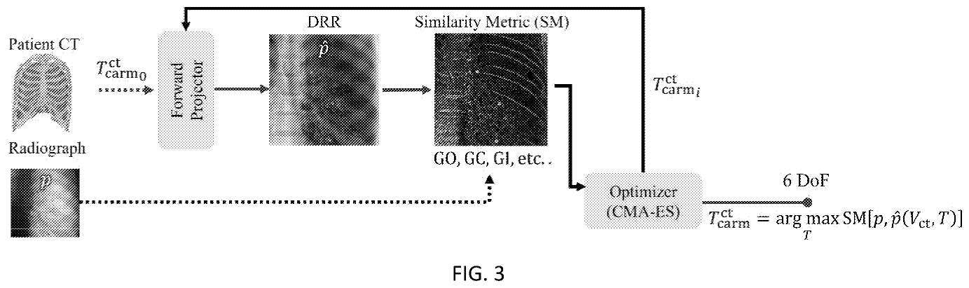

FIG. 3 illustrates a process of some embodiments for 3D-2D registration, suitable for rigid registration of high-contrast structures. In this example, a similarity metric (e.g., GO, GC, GI, or others) is computed between the DRR and projection and maximized via iterative optimization (e.g., CMA-ES, stochastic gradient descent, or other methods), yielding a 6 degrees-of-freedom (DoF) transform that gives the pose of the 3D model with respect to the imaging coordinate frame. Structures defined in the 3D image can thereby be overlaid in the projection radiograph.

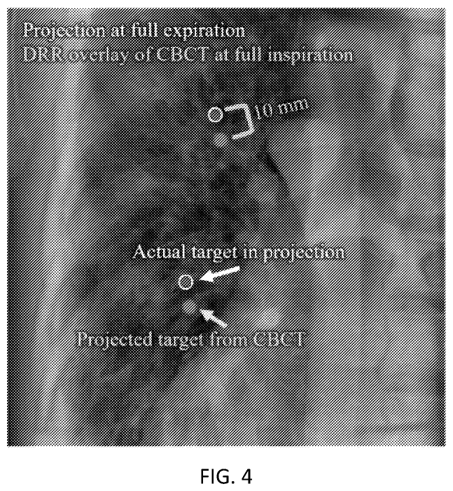

FIG. 4 illustrates a limitation in some embodiments of some 3D-2D registration methods: a conventional 3D-2D registration aligns the ribs well, but fails to register the underlying soft-tissue structures—e.g., a lung nodule target. The projection image is overlaid by (green) gradients from the DRR at solution. Because conventional 3D-2D registration is driven primarily by bone gradients, it exhibits good alignment of the spine and ribs, but low-contrast lung structures (e.g., a suspicious lung nodule that is the target of interest) are not well registered-due to both (1) distraction of the algorithm by bone gradients; and (2) a simple rigid transformation model that does not account for soft-tissue deformation. Each of these points is resolved by the approach of other embodiments described below.

Some embodiments of the current invention are directed to a method to: (1) solve 3D-2D registration of such low-contrast structures; and (2) resolve soft-tissue deformation. An embodiment of the method for low-contrast soft-tissue registration is described in Example 1 via modification of the objective function using high-contrast gradient down-weighting to suppress the influence of bones and accentuate the influence of soft-tissues.

This method is extended for some embodiments, as described in Example 2 (below) to a local registration that solves the 3D-2D registration (via the method of Example 1) in a multi-scale, coarse-to-fine registration method about a single target region of interest (robust to deformation).

Finally, the method is further extended for some embodiments, as described in Example 3 (below) to a deformable registration by solving the local 3D-2D registration over multiple regions of interest and combining the results to yield a globally deformable registration over the full image.

### Example 1: Modification of the Objective Function to Encourage Soft-Tissue-Driven 3D-2D Registration

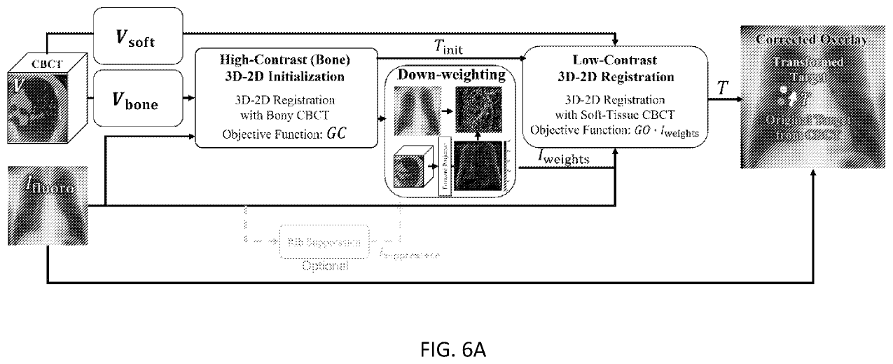

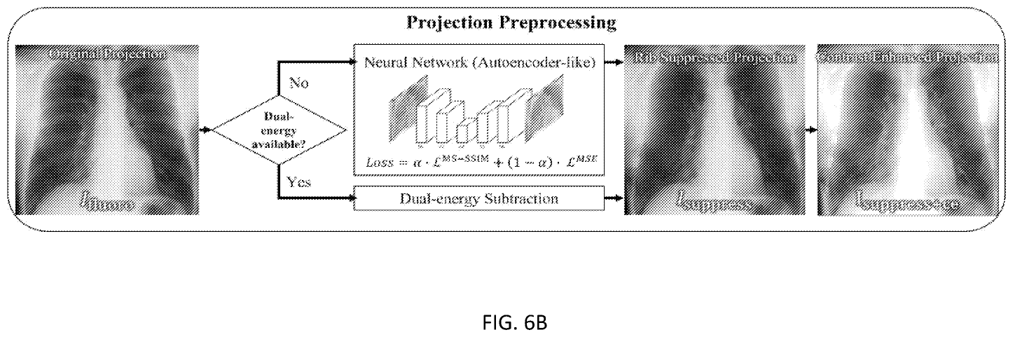

According to an embodiment of the current invention, 3D-2D registration of low-contrast soft-tissue structures within the context of high-contrast structures (bone or surgical instruments) is accomplished by suppressing the influence of strong gradients (e.g., bone) and/or enhancing the contribution of low-contrast gradients. This suppression is primarily achieved through a method that down-weights gradients belonging to high-contrast structures in the similarity map of the objective function. As illustrated in FIG. 6A (described below), the conventional similarity metric (e.g., GO or GC) presents as an “image” or “map” prior to summation. The proposed method “masks” regions of the similarity map associated with high-contrast bone gradients. This masking occurs in the similarity metric domain (which shares the coordinate frame of the projection domain). Gradients (and similarity metrics computed from such gradients, such as GO and GC) can be down-weighted to diminish their influence on the registration. Gradient down-weighting is applied to the similarity map as a weighting “mask” applied to the similarity metric map.

For instance, in the case of gradient orientation, defined below as

$$GO = \frac{1}{N} {\sum }_{i ∈ \{ Ω : {∇}_{u} {I}_{1} > {t}_{1} ⋂ {∇}_{u} {I}_{2} > {t}_{2} \}} p ( i ) \\ where p ( i ) = \frac{2 - ( ln ( \vert {cos}^{- 1} ( {α}_{i} ) \vert ) + 1 )}{2} \qquad ( 1 )$$

we can modify the similarity metric by including the down-weighting term

$$GO = \frac{1}{N} {\sum }_{i ∈ \{ Ω : {∇}_{u} {I}_{1} > {t}_{1} ⋂ {∇}_{u} {I}_{2} > {t}_{2} \}} p ( i ) {w}^{′} ( i ) \qquad ( 2 )$$

and w′ is the down-weighting map

To generate the gradient weighting map, an initial (conventional) 3D-2D rigid registration is first performed (driven by high-contrast bone gradients), and the map is derived from the DRR at solution. For example, a 3D-2D registration of a bone-thresholded CT registered to the radiograph yields the pose of bone structures (ribs), and the DRR generated from the CT after registration provides a map of gradients that are generated by ribs that can be used to derive the down-weighting map.

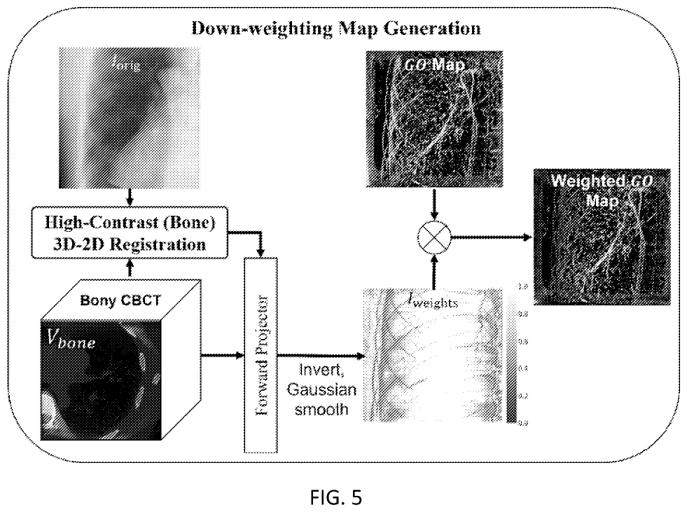

FIG. 5 shows a process of some embodiments and associated weighting maps for down-weighting of bone gradients, generated by solving a 3D-2D registration of high-contrast structures (e.g., bones) to the projection and inverting and smoothing the DRR at solution to generate a weighting mask, I_{weights}. The weighting mask is applied to the similarity map prior to summation (for example, to a gradient orientation or “GO” map), thereby yielding an objective function (similarity metric) that is less influenced by the presence of high-contrast bone structures. Note that the conventional “GO Map” presents strong gradients associated with the ribs and spine, whereas the “Weighted GO Map” diminishes the contribution of bones and emphasizes the gradients associated with soft-tissues (lungs and diaphragm), thereby driving the registration according to soft-tissues, instead of bones.

In some embodiments, other image processing steps are optionally applied in conjunction with gradient down-weighting as well. For example, low-contrast gradients can be enhanced using CLAHE and log normalization. Using these techniques, unlike in conventional approaches, the registration is driven primarily by low-contrast gradient content—e.g., the lung parenchyma and airways, which contain the targets of interest-rather than high-contrast bone gradients.

Initial implementation of the method according to some embodiments of the current invention is shown in FIG. 6A. The method has demonstrated success in initial laboratory experiments conducted in a ventilated, cadaveric specimen.

Although down-weighting is discussed here in accordance with some embodiments, other embodiments could instead, or in addition, include up-weighting.

FIG. 6A shows an implementation in some embodiments of the proposed method for low-contrast 3D-2D registration in pulmonary interventions. An initial 3D-2D registration of the bone-thresholded CBCT to the projection image to generates an initial transform Tᵢₙᵢₜ). A down-weighting mask is derived from the bone initialization and applied to the similarity map prior to summation to yield a novel similarity metric that emphasizes low-contrast gradients. In this example, gradient correlation was chosen for bony anatomy registration due to its robustness against content mismatch. Low-contrast 3D-2D registration proceeds by registering a soft-tissue-thresholded CBCT to the projection using the initial transform. The similarity metric used in this example is gradient orientation, which de-emphasizes gradient magnitude allowing lower-magnitude gradients to drive the registration. The low-contrast 3D-2D registration produces a transform, T, which can be used to correct the position of planning data in the CBCT. The corrected planning data can then be forward projected to fluoroscopic overlay.

An alternate method of some embodiments for gradient down-weighting for suppressing the influence of bone gradients in 3D-2D registration is shown in FIG. 6B, where rib suppression accomplished via neural network image processing. Additional suppression of strong bony gradients in the projection that do not belong to the target anatomy can be achieved in the projection domain. Two simple methods for doing so have been established in prior art: rib suppression image processing and dual-energy imaging. For example, neural networks for rib suppression have been demonstrated in diagnostic chest radiography for a very distinct purpose from that described here-namely, increased diagnostic accuracy in visualization of lung nodules that may be obscured by overlying ribs in the image [1]. Alternatively, bone structures can be suppressed in the projection domain by dual-energy subtraction [2]. Prior art describes each of these methods in the context of improved visualization in diagnostic imaging, and their adaptation to the context of 3D-2D registration of low-contrast soft-tissue structures is novel. This method can be used in some embodiments in conjunction with gradient down-weighting prior to 3D-2D registration.

### Example 2: Multi-Scale, Locally-Rigid 3D-2D Registration—with Application to Pulmonary Interventions

For 3D-2D registration in the presence of anatomical deformation, an embodiment of the current invention can include a multi-scale 3D-2D registration method that solves 3D-2D registrations in small (“local”) regions in the projection under the conventional rigid motion (6 DoF) assumption.

This method begins by designating a small region-of-interest (ROI) in the projection centered upon a feature of interest (e.g., a lung nodule). In Example 2, a single ROI is considered. In Example 3, the method is extended to multiple ROIs and a globally deformable solution. The registration is initialized by first performing a rigid global 3D-2D registration of the entire projection with coarse subsampling, followed by rigid 3D-2D registrations using progressively finer sampling and cropping of the projection centered around the target ROI. After a rigid solution has been found local to the ROI, the deformed position of objects designated in the 3D volume within the ROI can be overlaid on the projection.

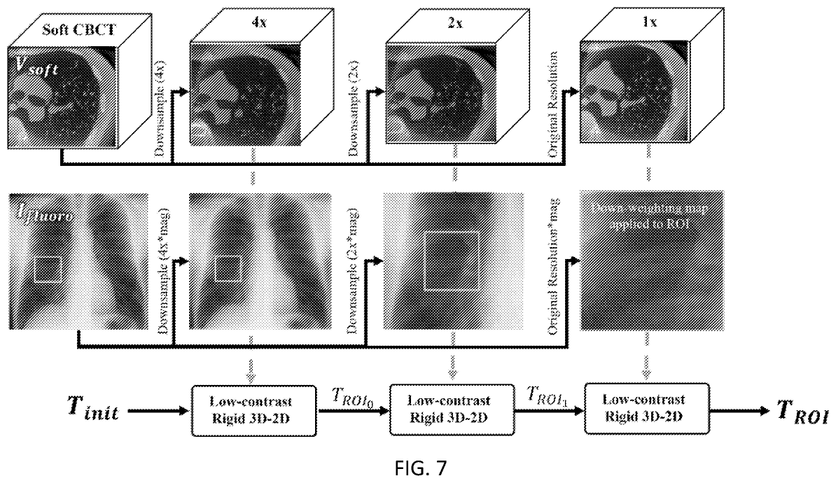

The method can be combined with those described in Example 1 (described above) for 3D-2D registration of soft-tissue structures, as shown below in FIG. 7.

FIG. 7 shows an example in some embodiments of a low-contrast, locally rigid 3D-2D registration for a designated ROI in a chest radiograph. A soft-tissue thresholded CBCT (V soft) is registered to the chest radiograph in a multi-scale approach in which the projection is sampled in a coarse-to-fine manner at each level, and the projection is increasingly cropped at each level to zoom in about the ROI. Gradient down-weighting is applied at the final level to de-emphasize strong rib gradients for low-contrast 3D-2D registration. The resulting transform can be used to update fluoro overlays within the ROI.

The methods described here are applicable for any similarity metric, but in the case of pulmonary interventions, the gradient orientation (GO) similarity metric is favored for low-contrast, locally rigid 3D-2D registration since it reduces the effect of large gradient magnitudes that can be produced by bone and extraneous instrumentation. The similarity metric was defined in equation (1), where t₁ and t₂ represent tunable gradient magnitude thresholds for the fixed and moving images, respectively. 3D-2D registration algorithms for bone anatomy use these thresholds to filter noisy and low-magnitude gradient content, using the median gradient magnitude value as the default thresholds of both images. For the purposes of pulmonary interventions, these thresholds have been tuned to allow low-magnitude gradients (in addition to gradient down-weighting).

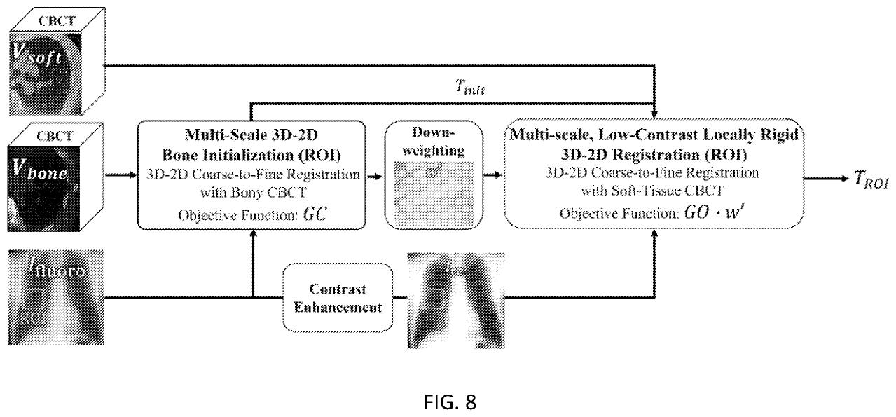

FIG. 8 shows an example of some embodiments of multi-scale, locally-rigid 3D-2D registration combined in some embodiments with methods (from Example 1) for low-contrast 3D-2D registration. Low-contrast 3D-2D registration at is computed at a local level via ROIs selected in the image corresponding to specific targets. A multi-scale 3D-2D registration is performed at the ROI using the bone-thresholded CBCT, with each scale getting progressively smaller around the ROI. The registration provides an initial transform Tᵢₙᵢₜ, as well as a weighting map for each ROI. Next, the projection is contrast-enhanced, and a low-contrast 3D-2D registration is computed at the ROI, generating a transform TROI which can be used to augment fluoroscopic overlay within the ROI.

A case example in pulmonary interventions is now considered. First, a CBCT is acquired of the patient at a fixed respiratory volume, and planning is performed on the volume (including designation of lung nodules, target ROIs, and trajectories). During the intervention, a fluoroscopic projection is acquired at a different respiratory volume. The task is to now estimate the lung deformation that has occurred to correct overlays of the lung nodules and trajectories on the projection while avoiding the confounding influence of the ribs.

For a given ROI, an initial transform is generated via multi-scale 3D-2D rigid registration of a bone-thresholded CBCT (V_{bone}—a threshold-based segmentation of the bony anatomy in the CBCT) to the fluoroscopic projection. Since the CBCT is bone-thresholded, the DRR at solution contains only a forward projection of bone within the local ROI, and the resulting DRR can be scaled and inverted to down-weight the regions of the projection corresponding to strong bone gradients, generating a weighting map which can be optionally used for low-contrast 3D-2D registration.

The projection is then contrast-enhanced for use in multi-scale, low-contrast 3D-2D registration at the ROI. To perform this registration, the CBCT is soft-tissue-thresholded (V soft), and the registration is initialized using the solution of the bone CBCT registration. The gradient down-weighting mask is applied to the similarity metric map during optimization, and the result is used to correct overlays within the ROI (e.g., lung nodule markers).

### Example 3: Extension of Locally-Rigid 3D-2D Registration to a Globally Deformable Solution

The method described in Example 2 can be applied to multiple ROIs that are designated either uniformly across the image or at specific target locations. These registrations (solved in parallel) provide transforms that can be used to augment fluoro overlay within each ROI (as previously mentioned) or can alternatively be used as control points in an interpolation to correct global overlays (e.g., trajectories), providing a global deformation vector field.

The method is analogous to a previously described method for multi-scale registration of vertebral labels [3]. The method described herein is novel and distinct, however, not only in that it exercises the methods from Example 1 (above) for registration of low-contrast, soft-tissue structures (cf., the spine) but also in that it effects a globally deformable registration that can be applied to the image as a whole (and not just individual point labels therein) via an interpolation of locally rigid registrations.

The combined method of some embodiments (Example 1—low-contrast registration) and (Example 2—multi-scale locally rigid 3D-2D registration) applied to many ROIs (Example 3—extension to global deformation) is illustrated in FIG. 8 to realize a potentially valuable framework for deformable 3D-2D registration and guidance of pulmonary interventions.

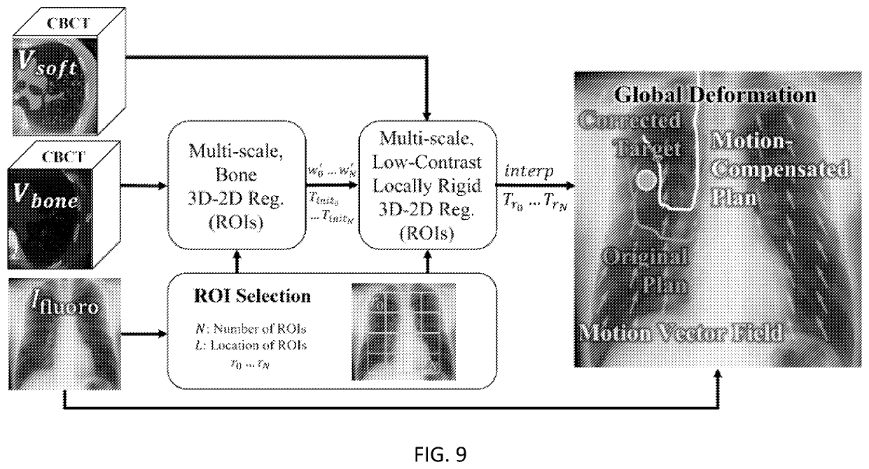

FIG. 9 shows an example of some embodiments of multi-scale, locally-rigid, globally deformable 3D-2D registration combined with methods (from Example 1) for low-contrast 3D-2D registration, multi-scale locally rigid 3D-2D registration (Example 2), applied to many ROIs (Example 3). ROIs are first selected on the projection (in this example, uniformly across the image). At each ROI, a multi-scale bone initialization is performed to generate an initial transform and weighting map. These are used to perform a multi-scale, locally-rigid 3D-2D registration at the ROI. This process is repeated in parallel for all designated ROIs, and the solution at each ROI is interpolated to create a deformation vector field which can be used to augment 3D plans throughout the image (e.g., a trajectory plan for a robotic system).

The system includes a number of components that each may be implemented on a server or on an end-user device. In some cases, a subset of the components may execute on a user device (e.g., a mobile application on a cell phone, a webpage running within a web browser, a local application executing on a personal computer, etc.) and another subset of the components may execute on a server (a physical machine, virtual machine, or container, etc., which may be located at a datacenter, a cloud computing provider, a local area network, etc.).

The components of the system may be implemented in some embodiments as software programs or modules, which are described in more detail below. In other embodiments, some or all of the components may be implemented in hardware, including in one or more signal processing and/or application specific integrated circuits. While the components are shown as separate components, two or more components may be integrated into a single component. Also, while many of the components' functions are described as being performed by one component, the functions may be split among two or more separate components.

In addition, at least one figure conceptually illustrates a process. The specific operations of this process may not be performed in the exact order shown and described. The specific operations may not be performed in one continuous series of operations, and different specific operations may be performed in different embodiments. Furthermore, the process could be implemented using several sub-processes, or as part of a larger macro process.

The terms “light” and “optical” are intended to have broad meanings that can include both visible regions of the electromagnetic spectrum as well as other regions, such as, but not limited to, infrared and ultraviolet light and optical imaging, for example, of such light.

The terms “computer”, “server”, “processor”, and “memory” all refer to electronic or other technological devices. These terms exclude people or groups of people. As used in this specification, the terms “computer readable medium,” “computer readable media,” and “machine readable medium,” etc. are entirely restricted to tangible, physical objects that store information in a form that is readable by a computer. These terms exclude any wireless signals, wired download signals, and any other ephemeral signals.

The term “computer” is intended to have a broad meaning that may be used in computing devices such as, e.g., but not limited to, standalone or client or server devices. The computer may be, e.g., (but not limited to) a personal computer (PC) system running an operating system such as, e.g., (but not limited to) MICROSOFT® WINDOWS® available from MICROSOFT® Corporation of Redmond, Wash., U.S.A. or an Apple computer executing MAC® OS from Apple R: of Cupertino, Calif., U.S.A. However, the invention is not limited to these platforms. Instead, the invention may be implemented on any appropriate computer system running any appropriate operating system. In one illustrative embodiment, the present invention may be implemented on a computer system operating as discussed herein. The computer system may include, e.g., but is not limited to, a main memory, random access memory (RAM), and a secondary memory, etc. Main memory, random access memory (RAM), and a secondary memory, etc., may be a computer-readable medium that may be configured to store instructions configured to implement one or more embodiments and may comprise a random-access memory (RAM) that may include RAM devices, such as Dynamic RAM (DRAM) devices, flash memory devices, Static RAM (SRAM) devices, etc.

The secondary memory may include, for example. (but not limited to) a hard disk drive and/or a removable storage drive, representing a floppy diskette drive, a magnetic tape drive, an optical disk drive, a read-only compact disk (CD-ROM), digital versatile discs (DVDs), flash memory (e.g., SD cards, mini-SD cards, micro-SD cards, etc.), read-only and recordable Blu-Ray R discs, etc. The removable storage drive may, e.g., but is not limited to, read from and/or write to a removable storage unit in a well-known manner. The removable storage unit, also called a program storage device or a computer program product, may represent, e.g., but is not limited to, a floppy disk, magnetic tape, optical disk, compact disk, etc. which may be read from and written to the removable storage drive. As will be appreciated, the removable storage unit may include a computer usable storage medium having stored therein computer software and/or data.

In some embodiments, the secondary memory may include other similar devices for allowing computer programs or other instructions to be loaded into the computer system. Such devices may include, for example, a removable storage unit and an interface. Examples of such may include a program cartridge and cartridge interface (such as, e.g., but not limited to, those found in video game devices), a removable memory chip (such as, e.g., but not limited to, an erasable programmable read only memory (EPROM), or programmable read only memory (PROM) and associated socket, and other removable storage units and interfaces, which may allow software and data to be transferred from the removable storage unit to the computer system.

Some embodiments include electronic components, such as microprocessors, storage and memory that store computer program instructions in a machine-readable or computer-readable medium (alternatively referred to as computer-readable storage media, machine-readable media, or machine-readable storage media). The computer-readable media may store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

The computer may also include an input device may include any mechanism or combination of mechanisms that may permit information to be input into the computer system from, e.g., a user. The input device may include logic configured to receive information for the computer system from, e.g., a user. Examples of the input device may include, e.g., but not limited to, a mouse, pen-based pointing device, or other pointing device such as a digitizer, a touch sensitive display device, and/or a keyboard or other data entry device (none of which are labeled). Other input devices may include, e.g., but not limited to, a biometric input device, a video source, an audio source, a microphone, a web cam, a video camera, and/or another camera. The input device may communicate with a processor either wired or wirelessly.

The computer may also include output devices which may include any mechanism or combination of mechanisms that may output information from a computer system. An output device may include logic configured to output information from the computer system. Embodiments of output device may include, e.g., but not limited to, display, and display interface, including displays, printers, speakers, cathode ray tubes (CRTs), plasma displays, light-emitting diode (LED) displays, liquid crystal displays (LCDs), printers, vacuum florescent displays (VFDs), surface-conduction electron-emitter displays (SEDs), field emission displays (FEDs), etc. The computer may include input/output (I/O) devices such as, e.g., (but not limited to) communications interface, cable and communications path, etc. These devices may include, e.g., but are not limited to, a network interface card, and/or modems. The output device may communicate with processor either wired or wirelessly. A communications interface may allow software and data to be transferred between the computer system and external devices.

The terms “processor,” “processing unit,” “data processor,” etc. are intended to have a broad meaning that includes one or more processors, such as, e.g., but not limited to, processors that are connected to a communication infrastructure (e.g., but not limited to, a communications bus, cross-over bar, interconnect, or network, etc.). The terms may include any type of processor, microprocessor and/or processing logic that may interpret and execute instructions, including application-specific integrated circuits (ASICs) and field-programmable gate arrays (FPGAs). The data processor may comprise a single device (e.g., for example, a single core) and/or a group of devices (e.g., multi-core). The data processor may include logic configured to execute computer-executable instructions configured to implement one or more embodiments. The instructions may reside in main memory or secondary memory. The data processor may also include multiple independent cores, such as a dual-core processor or a multi-core processor. The data processors may also include one or more graphics processing units (GPU) which may be in the form of a dedicated graphics card, an integrated graphics solution, and/or a hybrid graphics solution. Various illustrative software embodiments may be described in terms of this illustrative computer system. After reading this description, it will become apparent to a person skilled in the relevant art(s) how to implement the invention using other computer systems and/or architectures.

The term “data storage device” is intended to have a broad meaning that includes removable storage drive, a hard disk installed in hard disk drive, flash memories, removable discs, non-removable discs, etc. In addition, it should be noted that various electromagnetic radiation, such as wireless communication, electrical communication carried over an electrically conductive wire (e.g., but not limited to twisted pair, CAT5, etc.) or an optical medium (e.g., but not limited to, optical fiber) and the like may be encoded to carry computer-executable instructions and/or computer data that embodiments of the invention on e.g., a communication network. These computer program products may provide software to the computer system. It should be noted that a computer-readable medium that comprises computer-executable instructions for execution in a processor may be configured to store various embodiments of the present invention.

The term “network” is intended to include any communication network, including a local area network (“LAN”), a wide area network (“WAN”), an Intranet, or a network of networks, such as the Internet.

The term “software” is meant to include firmware residing in read-only memory or applications stored in magnetic storage, which can be read into memory for processing by a processor. Also, in some embodiments, multiple software inventions can be implemented as sub-parts of a larger program while remaining distinct software inventions. In some embodiments, multiple software inventions can also be implemented as separate programs. Finally, any combination of separate programs that together implement a software invention described here is within the scope of the invention. In some embodiments, the software programs, when installed to operate on one or more electronic systems, define one or more specific machine implementations that execute and perform the operations of the software programs.

### REFERENCES

[1] Gusarev, Maxim, et al. “Deep Learning Models for Bone Suppression in Chest Radiographs.” 2017 IEEE Conference on Computational Intelligence in Bioinformatics and Computational Biology (CIBCB), 2017, https://doi.org/10.1109/cibcb.2017.8058543.
[2] Kuhlman, Janet E., et al. “Dual-Energy Subtraction Chest Radiography: What to Look for beyond Calcified Nodules.” RadioGraphics, vol. 26, no. 1, 2006, pp. 79-92., https://doi.org/10.1148/rg.261055034.
[3] Ketcha, M D, et al. “Multi-Stage 3D-2D Registration for Correction of Anatomical Deformation in Image-Guided Spine Surgery.” Physics in Medicine and Biology, vol. 62, no. 11, 2017, pp. 4604-4622., https://doi.org/10.1088/1361-6560/aa6b3e.

The embodiments illustrated and discussed in this specification are intended only to teach those skilled in the art how to make and use the invention. In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described. For example, it is to be understood that the present disclosure contemplates that, to the extent possible, one or more features of any embodiment can be combined with one or more features of any other embodiment.

1. A method for registering a two-dimensional (2D) x-ray image to a three-dimensional (3D) x-ray image, comprising:
constructing said 2D x-ray image of an object of interest;
receiving said 3D x-ray image of said object of interest;
generating a plurality of 2D projection images from said 3D x-ray image of said object of interest for each of a corresponding plurality of different poses of said object of interest; and
registering said 2D x-ray image to one of said 2D projection images,
wherein said registering uses a similarity metric that is differentially weighted to affect an influence of at least one of low-contrast or high-contrast structures on the registering between said 2D x-ray image and each of said 2D projection images.

2. The method according to claim 1, wherein said similarity metric is based on at least one of image gradients, image intensities, or a statistical distribution thereof.

3. The method according to claim 1, wherein said weighted similarity metric weights at least one of image gradients, intensities, or a statistical distribution thereof according to a magnitude thereof.

4. The method according to claim 1, wherein said similarity metric is a gradient correlation (GC) similarity metric.

5. The method according to claim 1, wherein said similarity metric is a gradient orientation (GO) similarity metric.

6. The method according to claim 1, further comprising, prior to said registering, preprocessing said 2D x-ray image to remove high contrast gradient regions.

7. The method according to claim 1, further comprising:
selecting a subregion of said 2D x-ray image at a first subsampling;
registering said 2D x-ray image to one of said 2D projection images based on said selected subregion; and
repeating said selecting and said registering for a second subsampling that is finer than said first subsampling such that registering said 2D x-ray image to one of said 2D projection images so as to register a deformable structure.

8. The method according to claim 7, further comprising:
repeating said selecting a subregion a plurality of times for different subregions of said, each at a first subsampling; and
repeating said registering and said repeating said selecting and said registering for corresponding second subsampling that is finer than said corresponding first subsampling such that registering said 2D x-ray image to one of said 2D projection images for each selected subregion so as to register a deformable structure according to a deformation map across a region of said 2D x-ray image.

9. The method according to claim 1, wherein said constructing said 2D x-ray image of an object of interest comprises constructing a radiograph, and
receiving said 3D x-ray image of said object of interest comprises receiving one of a computed tomography (CT) x-ray image, a cone-beam CT x-ray image, or a fan-beam CT x-ray image.

10. The method according to claim 9, further comprising constructing said 3D x-ray image.

11. A computer-readable medium for registering a two-dimensional (2D) x-ray image to a three-dimensional (3D) x-ray image, the computer-readable medium comprising non-transient code which when executed by a computer causes the computer to:
construct said 2D x-ray image of an object of interest;
receive said 3D x-ray image of said object of interest;
generate a plurality of 2D projection images from said 3D x-ray image of said object of interest for each of a corresponding plurality of different poses of said object of interest; and
register said 2D x-ray image to one of said 2D projection images,
wherein said registering uses a similarity metric that is differentially weighted to affect an influence of at least one of low-contrast or high-contrast structures on the registering between said 2D x-ray image and each of said 2D projection images.

12-20. (canceled)

21. A system for registering a two-dimensional (2D) x-ray image to a three-dimensional (3D) x-ray image, said system comprising a processor and a memory, said memory comprising non-transient code which when executed by said processor causes the processor to:
construct said 2D x-ray image of an object of interest;
receive said 3D x-ray image of said object of interest;
generate a plurality of 2D projection images from said 3D x-ray image of said object of interest for each of a corresponding plurality of different poses of said object of interest; and
register said 2D x-ray image to one of said 2D projection images,
wherein said registering uses a similarity metric that is differentially weighted to affect an influence of at least one of low-contrast or high-contrast structures on the registering between said 2D x-ray image and each of said 2D projection images.

22-30. (canceled)

31. A two-dimensional (2D) x-ray system, comprising:
an x-ray illumination system constructed to illuminate an object of interest with an x-ray beam;
a detection system arranged to receive at least a portion of said x-ray beam after said x-ray beam after passing through at least a portion of said object of interest; and
a system for registering a two-dimensional (2D) x-ray image to a three-dimensional (3D) x-ray image, said system comprising a processor and a memory,
wherein said memory comprises non-transient code which when executed by said processor causes the processor to:
construct said 2D x-ray image of an object of interest;
receive said 3D x-ray image of said object of interest;
generate a plurality of 2D projection images from said 3D x-ray image of said object of interest for each of a corresponding plurality of different poses of said object of interest; and
register said 2D x-ray image to one of said 2D projection images,
wherein said registering uses a similarity metric that is differentially weighted to affect an influence of at least one of low-contrast or high-contrast structures on the registering between said 2D x-ray image and each of said 2D projection images.

32-40. (canceled)
